(19) United States
(12) Patent Application Publication
(10) Pub. No.: US 2005/0231186 A1
(43) Pub. Date: Oct. 20, 2005

(54) High throughput electrophysiology system

(76) Inventors: Rafael H. Saavedra Barrera, Santa Barbara, CA (US); Makoto Taketani, Irvine, CA (US)

Correspondence Address: MORRISON & FOERSTER LLP, 755 PAGE MILL RD, PALO ALTO, CA, 94304-1018, US

(21) Appl. No.: 11/086,181

(22) Filed: Mar. 21, 2005

Related U.S. Application Data
(60) Provisional application No. 60/555,756, filed on Mar. 23, 2004.

(51) Int. Cl. A61N 1/18; A61N 1/28

(52) U.S. Cl. 324/71.1

(57) ABSTRACT

A system and method for monitoring electrophysiological information from a tissue slice includes at least one probe having a plurality of electrodes. The system also comprises a controller configured to select tissue sites to be monitored and to be electrically stimulated. In one variation of the invention, a plurality of multi-electrode probes are managed by the controller. The system may further include a plurality of amplifier modules, one amplifier module associated with each probe. The amplifier module may serve a number of functions including amplifying electrical signals sensed by the electrodes, distributing stimulation signals to selected electrodes, and filtering signals evoked from the tissue sites. The system can provide automatic selection and switching of electrodes for monitoring and stimulating multiple tissue sites. Multiple probes, each adapted to monitor multiple tissue sites, may be associated with the controller such that multiple tissue slices may be interrogated in parallel.

### RELATED APPLICATIONS

This application claims priority to U.S. provisional patent application Ser. No. 60/555,756, filed on Mar. 23, 2004, which is hereby incorporated by reference in its entirety.

### FIELD OF INVENTION

This invention relates to a high throughput device capable of detecting, measuring, and recording electrical activity in large numbers of neurological tissues and slices. In one variation, the device may be considered a high-throughput electrophysiology recording system, particularly suitable for use in a laboratory.

### BACKGROUND OF INVENTION

Over the past decade or more, medical investigators have actively pursued the use of nerve cell and neuronal tissue electrical activity in assessing the effects of psycho-active materials on those tissues. When nerve cells are active, that activity is evidenced by generation of a potential or a voltage. This potential arises from changes in ion concentration inside and outside the cell membrane accompanied by a change in ion permeability in nerve cells. Measuring of this potential change and the ion concentration change (that is, the ion current) near the nerve cells with electrodes allows detection of nerve cell or tissue activity.

Early workers in the field measured this cell activity potential by inserting a glass electrode into an area containing cells to measure extracellular potential. When evoked potential due to stimulation was measured, a metal electrode for stimulation was inserted together with a glass electrode for recording. However, the insertion of these electrodes carried with it the possibility of causing cell damage and long term measurement was difficult to do. In addition, space restrictions and the need for operating accuracy then made multipoint simultaneous measurements difficult to achieve.

U.S. Pat. No. 5,563,067 issued Oct. 8, 1996; U.S. Pat. No. 5,810,725 issued Sep. 22, 1998; U.S. Pat. No. 6,132,683 issued Oct. 17, 2000; U.S. Pat. No. 6,151,519 issued Nov. 21, 2000; U.S. Pat. No. 6,281,670 issued Aug. 28, 2001; U.S. Pat. No. 6,288,527 issued Sep. 2, 2001; and U.S. Pat. No. 6,297,025 issued Oct. 2, 2001, (each to Sugihara et al and incorporated by reference) describe a device employing a planar electrode having a large number of microelectrodes on an insulated substrate that first allowed multi-point simultaneous measurements of potential change at a number of points This device had small electrode-to-electrode distances and allowed long-term measurement of neuronal electrical activity.

One commercially available device made according to the listed patents incorporated an integrated cell holding instrument having a planar electrode assembly having a plurality of microelectrodes and their respective lead-ins positioned on the surface of a glass plate. The electrode assembly often included half-split holders for fixing the planar electrode by holding it from the top and bottom. The holders were often positioned upon a printed circuit board

A typical planar electrode assembly was made up of a transparent Pyrex glass sheet having a thickness of 1.1 mm and a size of 50×50 mm. In the center of this substrate, 64 microelectrodes were formed in an 8×8 matrix. Each microelectrode was connected to a conductive lead-in. The exemplified electrodes were each 50×50 μm square (area 25×10² μm²) and the center-to-center distance between adjacent electrodes was 150 μm. Each side of the substrate had 16 contact points with a pitch of 1.27 mm, totaling 64 exterior contacts. These electric contact points were connected to the microelectrodes in the center of the substrate in a 1 to 1 correspondence.

These planar electrodes were manufactured in the following fashion. ITO (indium tin oxide), for example, was applied to form a layer of 150 nm thick on the surface of the glass plate used as the substrate. A conductive array was then formed using a photoresist and etching. On top of this layer, a negative photosensitive polyimide was applied to form a layer about 1.4 μm in thickness and then formed into an overlying insulative film. The ITO layer was then coated with nickel (15 to 500 nm thick) and gold (16 to 50 nm thick) in the microelectrode region and at the peripheral electric contact points. A cylindrical polymeric (e.g., polystyrene) frame having an inner diameter of 22 mm, an outer diameter of 26 mm, and a height of 8 mm was then stuck to the center of the glass plate using a silicone adhesive to form a cell holding part around the central part of 64 microelectrodes. The inside of this polystyrene frame was to be filled with solutions containing, e.g., chloroplatinic acid, lead acetate, and hydrochloric acid. Application of a modest electric current deposited platinum black gold plating of the microelectrodes.

The half-split holders were often molded of a resin having an arm portion for holding the edge of the planar electrode. Also, the upper portion of the holder was pivotable by an axis pin. The upper portion of the holder typically was equipped with control fixtures having 16×4 pairs of contacts. The contacts in the upper holder correspond to electric contact points of the planar electrode and were formed of a spring of metal such as BeCu coated with Ni and Au.

The pin parts protruding from the upper holder are alternately arranged so that the 16 pieces of the pin part protruding from the upper holder are lined in two staggered rows. This pin part is connected to a connector mounted on a printed circuit board used for connection with the outside.

Also, the spring contacts protrude from the bottom face of the upper holder. All contact the planar electrode with a predetermined contact pressure resulting in an electrical connection having only small contact resistance.

The printed circuit board serves not only for fixing the assemblies of the planar electrode and the holders but also provides an electrical connection (via a connector) to the outside, starting from the microelectrode of the planar electrode, via the conductive pattern, via the electric contact point, to the contact. Furthermore, the printed circuit board facilitates handling procedures, for example, in installation to the measurement apparatus.

The printed circuit board comprises a glass epoxy substrate having double-faced patterns and connectors at four parts around a circular opening formed in the center.

The printed circuit board usually has an edge part on each side with electric contact points to/on a double faced connector edge. For the purpose of assuring mechanical fixation, the upper holder can be fixed to the printed circuit board using, e.g., a clamp.

A configuration of a cell potential measurement apparatus using the above-configured integrated cell holding instrument includes an optical observation device such as an inverted microscope for optical observations of cells or tissues placed in the integrated cell holding instrument. The system may include one or more computers including a device for providing a stimulation signal to the cells and a device for processing an output signal from the cells. Finally, the device may have a cell culturing means for maintaining a suitable culture medium for the cells.

In addition to the inverted microscope a camera may also be included or used in place of a microscope. The system may include an image filing device. The camera may be a SIT camera. A SIT camera is a general term used for cameras which apply a static induction transistor to an image pickup tube, and a SIT camera is a representative example of very sensitive cameras.

A typical computer was a personal computer (for example, compatible with WINDOWS) having an A/D conversion board and measurement software. The A/D conversion board includes an A/D converter and a D/A converter. The A/D converter has 16 bits and 64 channels, and the D/A converter has 16 bits and 8 channels.

The earlier measuring software included software for determining conditions needed for providing a stimulation signal or recording conditions of an obtained detection signal. With this type of software, the computer was capable of structuring stimulation signals to the cells and processing the detected signal from the tissues or cells, and also was capable of controlling the optical observation devices (the SIT camera and the image filing device) and the cell culturing means.

The earlier software was reasonably flexible in that complicated stimulation conditions were possible (e.g., by drawing a stimulation waveform using the computer). The recording conditions included 64 input channels, a sampling rate of 10 kHz, and continuous recording over several hours. Selection of an electrode providing a stimulation signal or an electrode for a detection signal was specified, e.g., manually or using a computer mouse or pen. Also, various conditions such as temperature, pH of the cell culturing fluid, etc., were displayable.

The software provided a recording screen displaying a spontaneous action potential or an evoked potential detected in real-time at a maximum of 64 channels. The recorded or evoked potential was displayed on top of a microscope image of the tissue or cells. When the evoked potential was measured, the whole recording waveform was displayed. When the spontaneous action potential was measured, the recording waveform was displayed only when an occurrence of spontaneous action was detected by a spike detection function using a window discriminator or a waveform discriminator. When the recording waveform was displayed, measurement parameters (e.g., stimulation conditions, recording conditions, temperature, pH) at the time of recording was simultaneously displayed in real-time.

The software included data analysis, e.g., FFT analysis, coherence analysis, or another analysis software. In addition, the software had other functionalities, such as a single spike separation using a waveform discriminator, a temporal profile display function, a topography display function, and an electric current source density analysis function. Results of these analyses were displayable on top of the microscope image stored in the image filing device.

When a stimulation signal was emitted from the above-configured computer, the stimulation signal was forwarded by way of a D/A converter and an isolator to the cells or tissues. An evoked potential arising between microelectrodes and a ground level (potential of culture solution) is routed to the computer via 64 channels of a sensitized amplifier (for example, “AB-610J” manufactured by NIHON KODEN CO., LTD.) and an A/D converter. The amplification factor of the amplifier was 100 dB, and the frequency band was from 0 to 10 kHz. However, when an evoked potential by a stimulation signal was measured, the frequency band was selected to be from 100 Hz to 10 kHz using a low cut-off filter.

The tissue or cell culturing component had a temperature adjuster, circulator for culture solution, and supply of mixed gas of air and carbon dioxide.

Another form of the stimulation signal was a bipolar, constant voltage pulse having a pair of positive and negative pulses for eliminating artifacts, that is, for preventing DC components from flowing. A preferred stimulation signal was a positive pulse with a pulse width of 100 μs, an interval of 100 μs, and a negative pulse of 100 μs. The peak electric current of the positive-negative pulse was in the range of 30 to 200 μA.

The cell culturing means, when placed in the measurement apparatus, enabled continuous measurement over a long period of time. Alternatively, the integrated cell holding instrument allowed culturing separately from the measurement apparatus.

By using the above-mentioned cell potential measurement apparatus, nerve cells and organs were cultured on the integrated cell holding instrument and the potential change accompanied by activities of the nerve cells or nerve organs measured. The cerebral cortex section of rats were often used as nerve tissue.

Despite the flexibility of this device and the associated software, the overall ability of the earlier devices to provide high throughput sampling and selection of adaptive testing regimes was nonexistent.

There has been considerable effort to develop higher throughput methods and devices for electrophysiological recording from cells. These are particularly important in drug development where hundreds or thousands of compounds are to be electrophysiologically tested against cells or tissues. Recent developments in this field include high-throughput whole cell clamping using planar electrode, auto patch clamping using robotics, and high-throughput oocyte voltage clamping using robotics. These so-called cell based electrophysiological assays will definitely accelerate early stages of the drug development pipeline, however, tissue based (or slice) physiology is still necessary to determine psycho-active effects in intact tissues and to understand the compounds' mechanism of action. Dispersed cells are comparatively easier to handle, because they can be handled as solutions. Many types of dispensers are available to transfer cells. In contrast, nerve tissues and slices are very difficult to handle and are not homogeneous. The characteristics of tissues and slices require expert physiologist to run even simple experiments and also inhibit developing higher throughput system.

Recent development of planar electrode array systems have made slice physiology experiments somewhat more available to less skilled researchers by, for example, removing steps such as electrode preparation and searching for stimulating and recording sites. However, it still generally requires one physiologist to operate the system.

The device and procedures described here allow computer-controlled switching of electrode stimulation sites. In earlier systems, even in those where it is possible to stimulate from different sites, a human operator has been needed to move a physical connection (using a cable or similar hardware) from one site to another. This process is illustrated in FIG. 1.

As shown in FIG. 1, tissue slice physiology experiments have three main steps: (1.) an operator places the selected brain slice on the multi-electrode probe (1-2 minutes) (2.) the operator and the computer select the stimulation sites and configure the stimulation parameters (10 minutes). This step consists of repeating the following two sub-steps: (a.) the software stimulates brain slice, captures and analyzes data, and displays results to the operator; and (b.) the operator makes a change to the stimulation site on the slice and adjusts stimulation parameters. Finally, in step (3.), the experiment is conducted which can take, e.g., between 60 and 120 minutes.

Once appropriate stimulation sites on the slice and stimulation parameters have been found, it is possible to automate the running of an experiment by using specialized software and expert systems technology. However, without having a computer-controller hardware allowing the arbitrary selection of stimulation sites, step (2.) above cannot be automated.

Indeed, the number of experiments that can be run by a single operator is limited by the length of time required to carry out steps (1) and (2). This is illustrated in FIG. 2, where ‘O’ represents the time to physically place the brain slice on the experimental setup, ‘C’ represents the time required to select and otherwise, to configure the stimulation site and stimulation parameters, and ‘E’ represents the time to execute the experiment. As shown in FIG. 2, selection of the stimulation sites and parameters are performed manually by the operator. This significantly impedes experiment throughput.

None of the commercially available systems provide for automation of the configuration step as described herein.

### SUMMARY OF THE INVENTION

A system for monitoring electrophysiological information comprises at least one multi-electrode probe for monitoring and for stimulating tissue sites of a tissue sample placed on the probe. The system additionally includes a controller configured to select the tissue sites to be monitored and stimulated. In one variation of the invention, the controller is configured to automatically select the tissue sites to be monitored and stimulated.

The system may also comprise an amplifier module that is associated with each probe. In one variation the amplifier module is configured to amplify electrical signals evoked from the tissue sites. In another variation, the amplifier module is configured to distribute stimulation signals to the electrodes of the associated probe. The amplifier may thus work in combination with the controller to manage the probes and electrodes.

The controller may be configured to select a wide variety of stimulating signals and tissue sites. In one variation, different voltage potentials are applied to the tissue sites. In another variation, a constant voltage potential is applied to different sets of electrodes. The stimulation signal can be switched from a first tissue site to second tissue site within a predetermined time period such as, for example, 0.5 to 2.5 ms. The stimulation signal may be time modulated such that evoked electrical signals corresponding to each stimulation signal may be separately monitored or recorded.

The system may further comprise a computer connected to the controller. The computer may serve a variety of functions and is typically adapted to program the controller to automatically select tissue sites to be monitored and to deliver a preselected stimulation signal to an electrode set. The computer may also receive and record the electrical signals arising from the monitored tissue sites. Such signals include electrical signals evoked from tissue sites being stimulated as well as spontaneous action signals arising from tissue sites receiving little (or no) electrical stimulus. Indeed, a tissue site may be monitored that presents no electrical activity.

The probe may vary in size and structure. In one variation, the probe comprises a well having a planar base portion. The well is adapted to contain a tissue slice such as a brain tissue slice of a rat. The base portion supports the plurality of electrodes. There may be greater than 16 or perhaps, greater than 64 electrodes associated with each probe. Additionally, multiple probes may be connected to the controller such that multiple experiments may be run in parallel.

A method for monitoring electrophysiological information comprises (a.) placing a sample of tissue on a multi-electrode probe; (b.) selecting a first set of electrodes to monitor electrical activity of the tissue; (c.) automatically selecting a second set of electrodes to monitor electrical activity of the tissue; and (d.) monitoring the electrical activity. The tissue may be a tissue slice such as a brain slice of a mammal.

In a variation of the invention, the method further comprises selecting tissue sites to be stimulated with stimulation signals. The stimulation signals may be varied or identical. Also, the tissue sites or locations to receive stimulation signals may be varied. The first stimulating signal may be applied prior, simultaneous, or subsequent to the application of a second stimulating signal. In one variation, at least 64 stimulation signals are sequentially applied to different sites of the tissue sample.

The step of selecting electrodes to monitor may be carried out using a controller. The controller may also be configured to select the stimulation signals and to select the tissue sites to be stimulated.

The method may additionally comprise the step of amplifying each signal monitored. An amplifier module may be provided to amplify the evoked signals as well as to distribute the selected stimulation signal to selected electrodes of the probe.

In one method, tissue samples are placed in a plurality of multi element probes that are collectively managed by a controller. An amplifier module as described above may be provided to manage each probe. The amplifier module distributes stimulation signals selected by a controller to each probe and each electrode of that probe. In this manner, a plurality of tissue samples may be interrogated in parallel and automatically.

Another electrophysiological information monitoring system comprises at least one probe means for monitoring electrical activity of one or more tissue sites of a tissue sample and a control means connected to the probe means. The probe means generally comprises a plurality of multielectrodes. The control means is configured to automatically select electrodes for monitoring tissue sites. The control means may also be configured to select electrical stimulation signals to send to the probe means. Also, an amplifier means may be provided for each probe means such that electrical signals sensed from each microelectrode can be amplified. The amplifier means may also be configured to distribute the stimulation signals from the controller to the microelectrodes of the probe means. The system may also comprise a computer connected to the control means to supply commands to the control means for monitoring and stimulating the tissue sites. The computer may also be configured to monitor and/or record the electrical signals from the tissue sites being monitored.

In another variation of the invention, a system for monitoring electrophysiological information comprises a plurality of probes each adapted to hold a tissue slice. The probes include a plurality of electrodes that can monitor electrical activity of tissue sites of the tissue slice when the tissue slice is placed on the probe. The system further includes a daughter amplifier module for each probe. The daughter amplifier module is adapted to amplify signals arising or evoked from the tissue sites. The system further includes a plurality of daughter controllers to manage the daughter amplifier devices. A primary controller is configured to manage all the daughter controllers.

Aspects of the invention may vary. The probe may comprise at least 16 electrodes. In another variation, the probe comprises at least 64 electrodes. The system may also comprise between 4-10 probes for each daughter controller.

An integrated housing may contain the controllers and amplifiers. However, the probes are typically separated from the housing. Also, a computer may be connected to the primary controller. The computer is configured to provide instructions to the primary controller to select the electrical stimulation signals as well as to determine which tissue sites shall be monitored. The controller may be configured to time-multiplex the stimulation signals. In this manner, many tissue sample experiments may be run in parallel and analyzed simultaneously.

### BRIEF DESCRIPTION OF THE DRAWINGS

FIG. 1 is a block diagram illustrating the steps of an early multi-electrode system showing that an operator makes changes to the system settings before the actual experiment can commence.

FIG. 2 is an illustration showing a procedure in which an operator must configure and reconfigure the stimulation sites and stimulation parameters prior to beginning the actual experiment.

FIG. 3 is a block diagram illustrating the steps of a multi-electrode system having a controller for automatically selecting the stimulation sites and configuring the stimulation parameters.

FIG. 4 is an illustration showing a procedure in which the stimulation sites and stimulation parameters are automatically configured prior to beginning the actual experiment.

FIG. 5A is a block diagram of a multi-electrode device architecture.

FIG. 5B is a block diagram of a multi-electrode device architecture having two levels of controllers.

FIG. 6 is a block diagram of a controller.

FIG. 7 shows exemplary circuit diagrams for the controller digital motherboard.

FIGS. 8A-8B are exemplary circuit diagrams for controller daughter boards.

FIG. 9 is a block diagram of an amplifier module.

FIG. 10 is an exemplary circuit diagram for an amplifier digital motherboard.

FIGS. 11-12 are exemplary circuit diagrams for amplifier daughter boards.

### DESCRIPTION

Central to the system described here and the process of using it is the placement of a controller between a computer and a multielectrode probe for monitoring electrophysiological activity of a tissue slice placed on that probe. In particular, the controller switches (or selects) the electrodes to sense electrical activity at various tissue sites of the tissue slice. The controller may also be configured to activate one or more electrodes with a stimulating signal, thereby stimulating corresponding tissue sites. A computer is typically used to instruct or program the controller to carry out the selecting and switching process. Also, because of the low level of signal found in the neural tissue, an amplifier module is preferably introduced between each probe and the controller to amplify or otherwise condition the signals arising from the tissue.

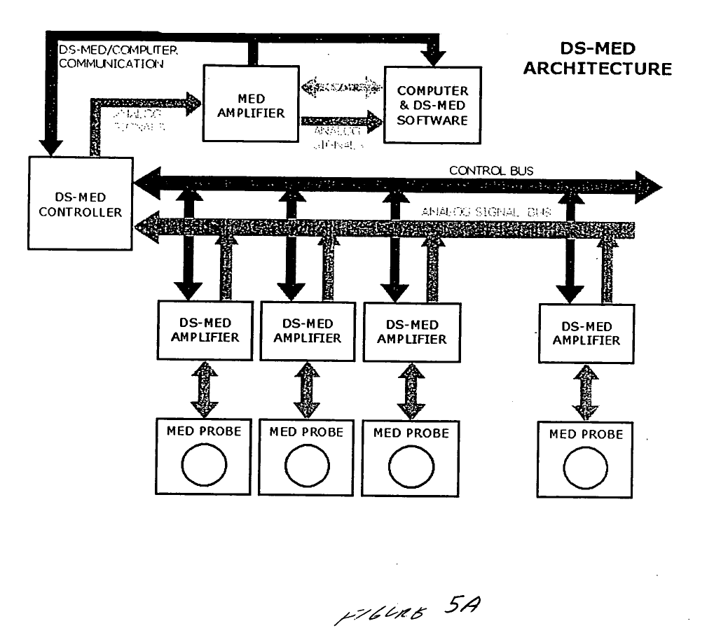

FIG. 3 shows in block diagram fashion, the manner in which the controller allows the computer to aid the analyzer and operator to choose appropriate slice position and parameters according to the particular analysis desired. The controller selects the tissue sites to monitor and stimulate without input from the operator.

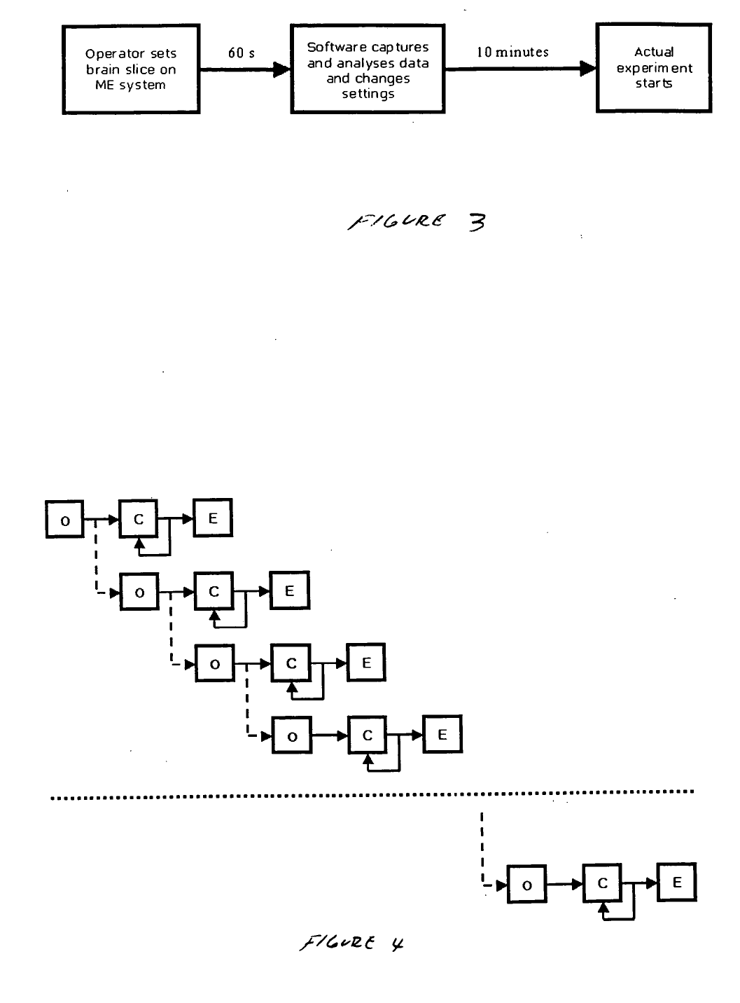

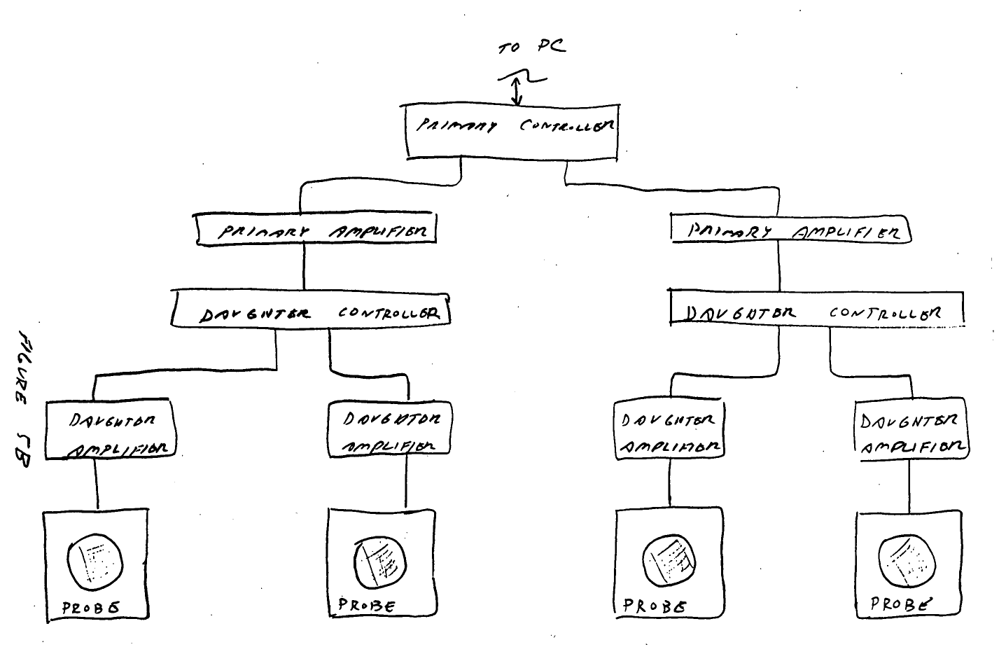

As shown in FIG. 4, the use of a controller to select a specific probe to be measured and software to measure, to compare, and to select (or not) the specific probe and as necessary, to adapt the stimulation parameters for a specific site, allows elimination of the manual review and selection step shown in FIG. 2. In particular, FIG. 4 shows that, in a situation where a series of experiments are to be run, the operator or technician performs only the initial set-up. In these types of experiments, the experience hand of the operator needed to set up the experiment is substantially lessened.

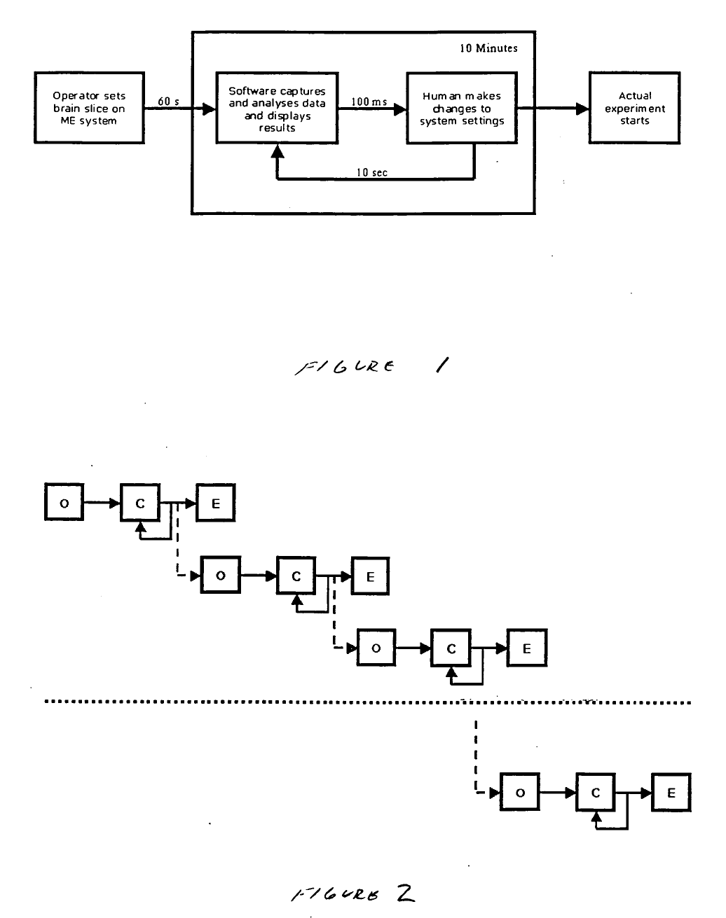

For example, assuming that on average it takes one minute to physically place a brain slice on the experimental platform and 10 minutes to select stimulation sites and to configure stimulation parameters, then using the device offered in FIG. 1 requiring human intervention during the configuring phase, the maximum number of experiments that a single technician can start is no more than six per hour. Using the system described in FIGS. 3-4, on the other hand, it would theoretically be possible to start up to 60 experiments per hour since the time-limiting step for the technician is the step of placing the brain slice on the right place of the multi element probe indicated by ‘O’ in FIG. 4.

Use of the procedures and devices described just above further allows the implementation of highly complicated and sophisticated protocols. For instance, the design of protocols, for instance, those requiring complex stimulation patterns, in which the stimulation of several independent sites is needed, may be achieved with but a small time delay between stimulations. In early multi-electrode physiological tissue monitoring systems, due to the fact that a human operator is required to set the stimulation site, the shortest time between stimulations is limited by the speed of the human in doing the actual switching from one site to another. Using the described devices and procedure, on the other hand, the switch may be made in milliseconds, making it possible to observe the evoked response of a network of neurons when those neurons are stimulated from different sites within a short time period. Since such brain natural phenomena occurs within the time constraints of the natural events in the brain, it is important to be able to mimic the same complex stimulation patterns in order to investigate realistic behavior.

The described procedure and hardware may be used to significantly reduce the cost of achieving high throughput on multi-electrode experiments by reducing the number of hardware elements. In using the procedure shown in FIG. 1, a conventional architecture, to increase throughput by a factor of N×N independent systems, each requiring a computer, amplifier, and additional devices, as well as some number of technicians are needed. Using the procedures and designs corresponding to FIGS. 3 and 4, on the other hand, reduces the complexity and cost by requiring but a single computer and simple modules, all potentially managed by a single technician.

Multi-experiment studies, for example, dosage-response studies may be optimized by, for instance, combining the results of several experiments running on a single system using the described procedures and devices and reconfiguring each experiment as a function of the results of experiments.

A basic system, not enhanced using the instant device and procedure, includes a multi-electrode array (a “MED-Probe”), an analog amplifier (a “MED-amplifier”), and the computer containing the analog-to-digital connector (“A/D converter”) and appropriate software.

The system described herein comprises two basic hardware components: a DS-MED controller and a dedicated DS-MED amplifier module for each probe. The DS-MED amplifier module may be, as described further below, an amplifier device comprising multiple circuits and boards for receiving, distributing, and/or conditioning signals.

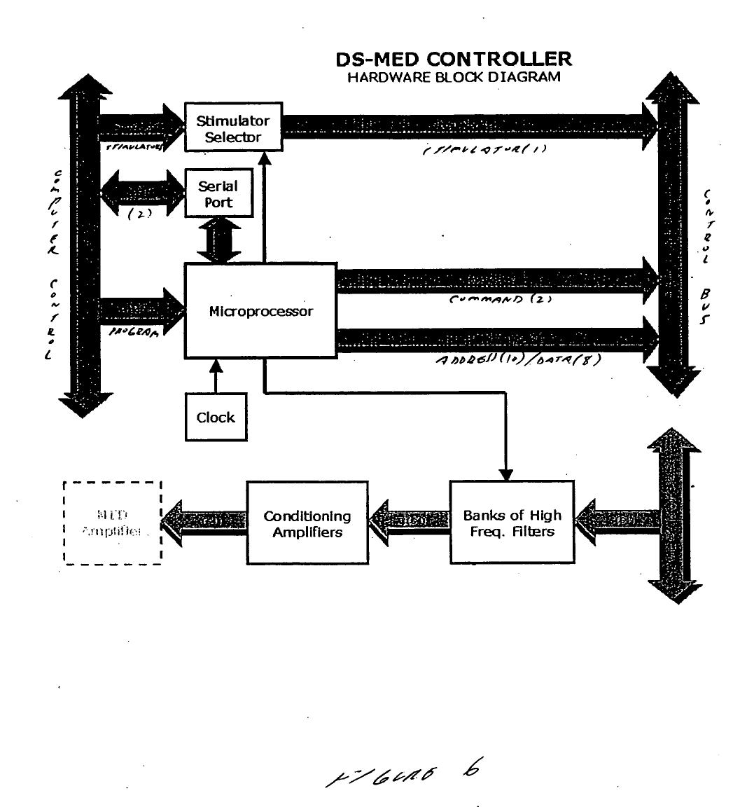

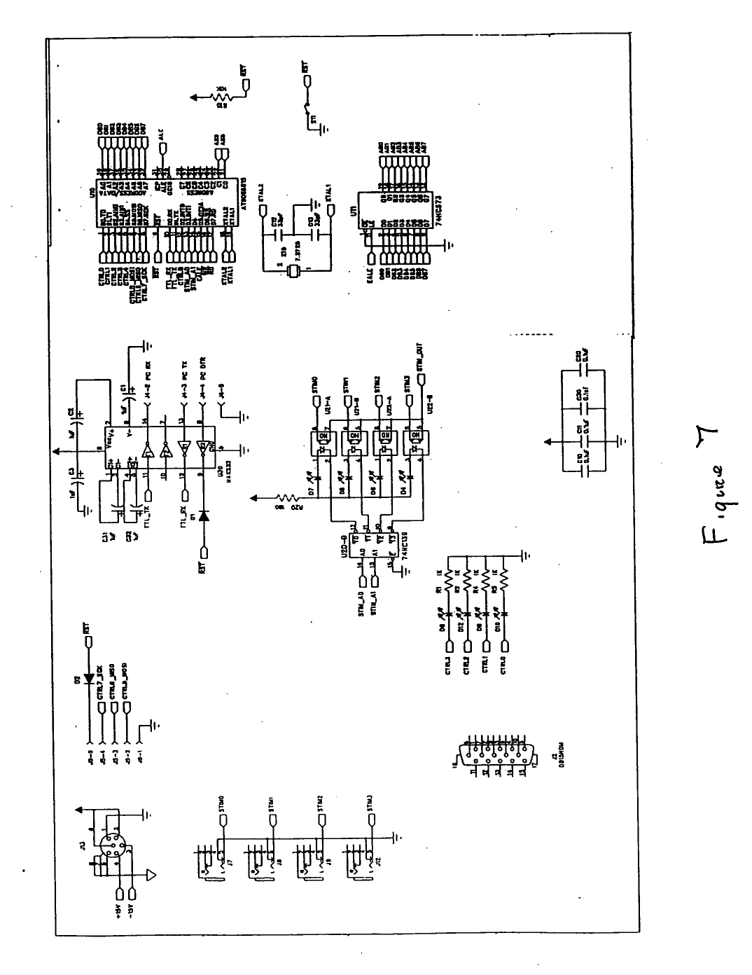

The DS-MED controller is connected to the system amplifier and emulates the behavior of a MED probe connected directly to the system amplifier. The controller is also connected to two buses, a Control Bus and an Analog Signal Bus. If a controller switches amongst, e.g., eight channels, each switched channel will include a DS-MED amplifier module for each probe. The control bus selects a probe or channel amongst those accessed by the DS-MED controller. Once so selected, the signal emitted by the probe is amplified by the channel DS-MED amplifier module, the so-amplified signal passes through the analog signal bus, passes through the DS-MED controller, the MED or system amplifier and onto the computer. Both the control bus and the analog bus are shared with each of the accessed DS-MED amplifier modules. In addition to being connected to the control and signal buses, the amplifier modules are directly connected to the MED probes. As shown in FIG. 5a, each amplifier module manages a single probe.

Furthermore, each probe includes a plurality of electrodes. The electrodes sense electrical activity in a tissue slice placed on/in the probe. The electrodes or tissue sites may be activated/stimulated by, for example, sending a stimulation signal to the electrodes. For example, a voltage potential may be applied between two or more electrodes. The DS-MED controller can be configured to automatically select electrodes to monitor and to activate. In this manner, the electrode monitoring and stimulating parameters may be configured for experiments relatively quickly. Additionally, by connecting multiple probes to a single controller and computer, multiple tissue slice experiments may be configured and run in parallel using, for example, time-multiplexing software.

In more complex configurations a primary amplifier can be used to manage one or more daughter controllers, which daughter controllers in turn can be connected to a group of amplifiers, and so on. In this way hierarchical configurations of many controllers and probes can be built. FIG. 5b shows an example of a multi-level DS-MED architecture.

Further details of the DS-MED controller and amplifier module are described below.

The DS-MED Controller

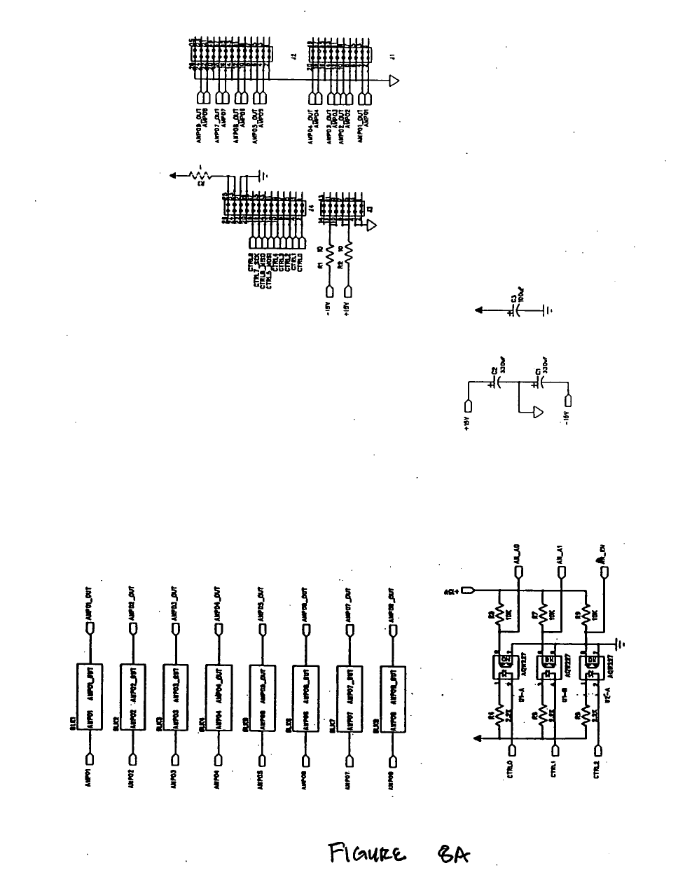

The DS-MED controller depicted in FIG. 6 includes 10 circuits: one digital motherboard, one analog motherboard, and eight identical 8-electrode filtering banks daughter boards. A block hardware diagram is presented in FIG. 6.

The digital motherboard contains a microprocessor running a low-level program for controlling the DS-MED amplifier modules which are connected to the control and analog signal busses as well as the communication with the DS-MED software running on the computer. The microprocessor sends commands, addresses, and operands to the DS-MED amplifier modules through the control bus. It also manages the communication to the computer and implements commands sent to it by the latter. These commands are used to 1) configure the individual DS-MED amplifier modules, 2) select one of the available stimulation sources (e.g., there may be four or more sources), and 3) select the specific high frequency filter on the eight 8-electrode daughter boards. As shown in FIG. 6, a serial port, a clock and the stimulation selection circuitry can also be included in the DS-MED controller.

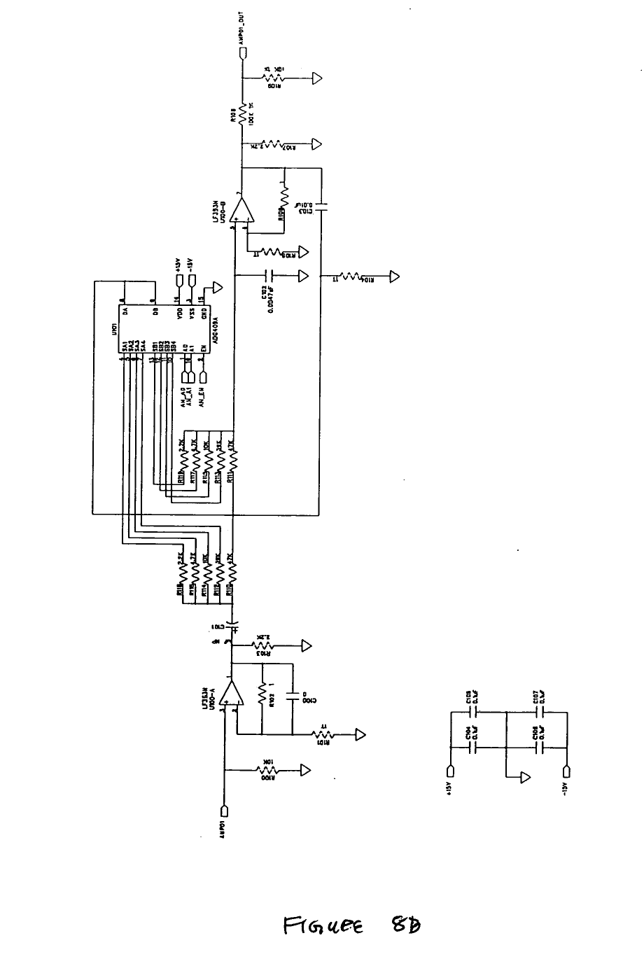

Finally, an interface may be included in the controller makes it possible to download new versions of the low-level program to run in the microprocessor and in this way reprogram and extend the functionality of the DS-MED controller in particular and the DS-MED architecture in general. An example circuit diagram is shown in FIG. 7.

The analog motherboard contains the interface between the analog signal bus and the inputs to the 8-electrode daughter boards, and between the output of these and the connector to the MED or system amplifier.

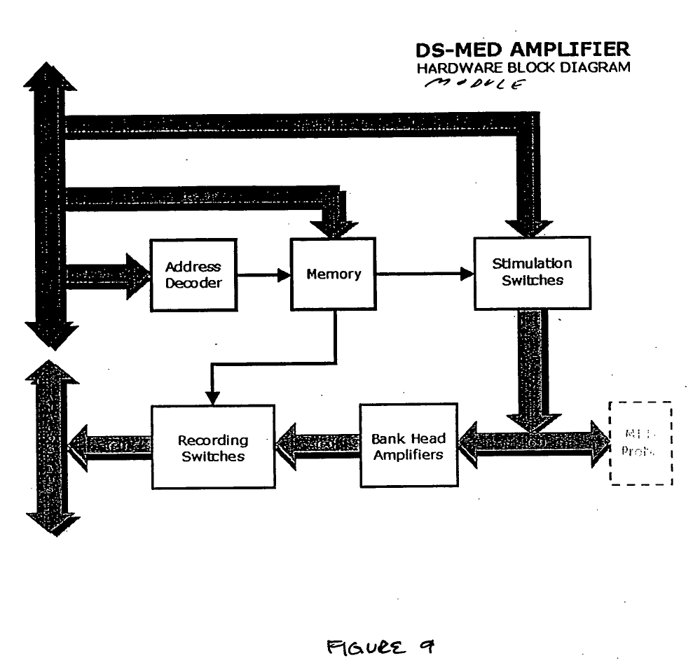

The eight 8-electrode daughter boards may each contain a set of high frequency filters and conditioning amplifiers, one set for each electrode. The filters are used to allow the A/D data acquisition cards to sub-sample the electrophysiological signals and in this way reducing the amount of data that has to be stored per experiment. The conditioning amplifiers make it possible to match the electrical characteristics of the analog signals to the requirements of the MED amplifier. Example circuit diagrams are shown in FIGS. 8A and 8B.

The DS-MED Amplifier

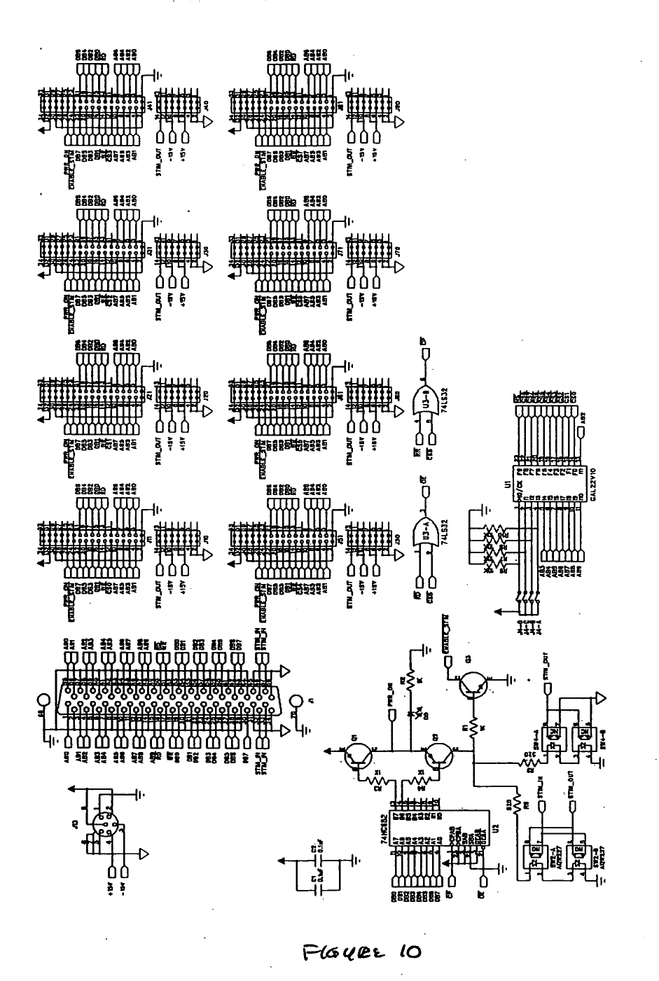

The DS-MED amplifier module shown in FIG. 9 may have 10 circuits: a digital motherboard, one analog motherboard, and eight daughter boards. A DS-MED amplifier block diagram is shown in FIG. 9.

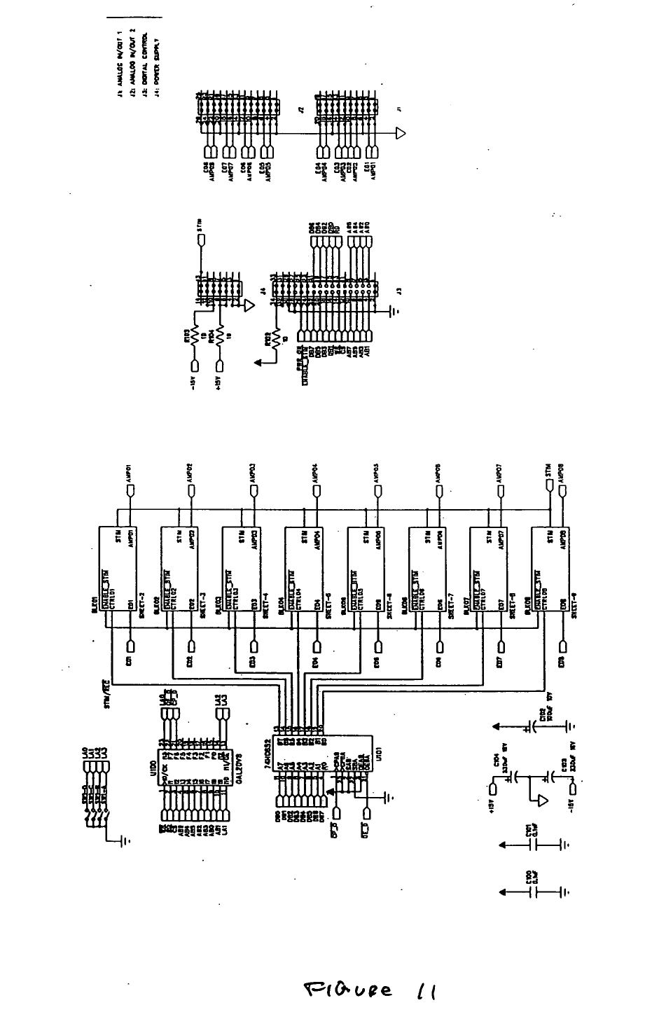

The digital motherboard for the DS-MED amplifier module typically includes circuitry to 1) identify uniquely each amplifier, 2) decode the address sent from the controller, 3) respond to the read and write commands from the controller, 4) maintain the state of probe, and 5) distribute the stimulation signal to the electrodes (e.g., to the 64 electrodes) through their respective daughter boards. A corresponding circuit diagram is shown in FIG. 10.

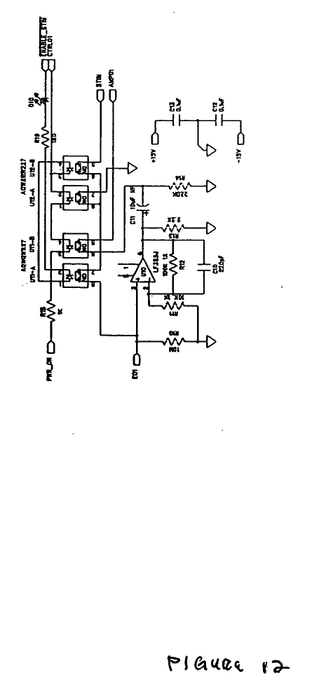

The analog motherboard for the DS-MED amplifier module typically includes an interface between the MED probe and the inputs to the 8-electrode daughter boards, and between the output of these and the analog signal bus. An example circuit diagram is shown in FIG. 11.

The eight 8-electrode daughter boards contain a bank of head amplifiers that condition the analog signals coming from the MED probes in order to transfer them without significant distortion to the analog signal bus. There is also circuitry to allow each electrode to function either as a recording or a stimulation electrode and to transfer the stimulation signal to the probe. The circuit diagrams are shown in FIGS. 11-12.

The above described DS-MED architecture provides for a number of advantages and benefits. The described procedure may provide, for example, flexible architecture scales. That is to say that a wide variety of systems are possible using the described devices and procedures. For instance, the described device may be used to build a simple single probe, or a 1-dimensional system with N probes, or more complex systems, e.g., two dimensions in which a controller manages several 1-dimensional systems, each with some number of probes. The described system may also provide modularity. By combining the described components, we are able to build a system having an arbitrary number of probes under the control of a single computer or several systems each with a smaller number of probes, each connected to a single computer. This system may further provide automatic selection of one of a plurality of MED amplifier stimulators (e.g., 4) and one of a plurality of MED probe electrodes (e.g., 64) as a target site for stimulation. Additionally, all experiments may run at the same time by time-multiplexing the use of available MED probes which may be carried out under the control of software.

Suitable software may be that known or readily developed by those of ordinary skill in the art to carry out the procedures and systems described here. Preferably, the software provides a convenient user-interface to control selection of electrodes and tissue sites to be monitored and activated. For example, the software may run a procedure that arbitrarily monitors each and every site as well as stimulates each and every site with various stimulation signals. The software also preferably facilitates the recording and analyzing of information. For example, the software may run an algorithm that compares measured signals to a threshold value. Still other suitable software may be used with the hardware described here.

The inventive system and procedure provides still other advantages and benefits. The invention may be embodied in other forms without departing from the spirit or essential characteristics thereof. The embodiments disclosed here are to be considered only as illustrative and not as restrictive. The scope of the invention is found in the appended claims; all changes which come within the meaning and range of equivalency of the claims are intended to be embraced therein.

1. A system for monitoring electrophysiological information comprising:
at least one probe comprising a plurality of electrodes for monitoring electrical activity of a plurality of tissue sites of a tissue sample placed on the probe; and
a controller configured to select at least one of said electrodes for monitoring the electrical activity at one or more of the tissue sites.

2. The system of claim 1 wherein said controller is further configured to provide a stimulation signal to at least one of said electrodes to stimulate at least one of said tissue sites.

3. The system of claim 2 comprising an amplifier module associated with each probe, said amplifier module configured to amplify electrical signals evoked from said tissue sites.

4. The system of claim 3 wherein said amplifier module is further configured to distribute said stimulation signal to the electrodes of the at least one probe.

5. The system of claim 2 further comprising a computer connected to said controller, said computer being adapted to program said controller to automatically select tissue sites to monitor and to deliver a selected stimulation signal to said tissue sites.

6. The system of claim 2 wherein said controller selects a first voltage potential to be applied between a first set of electrodes and, within a predetermined time period, selects a second voltage potential different than said first voltage potential to be applied between said first set of electrodes.

7. The system of claim 6 wherein said first set of electrodes is a pair of electrodes.

8. The system of claim 6 wherein said predetermined time period is 1 ms.

9. The system of claim 1 wherein said controller selects a first voltage potential to be applied between a first set of electrodes and subsequently selects said first voltage potential to be applied between a second set of electrodes wherein said first set of electrodes is not identical to said second set of electrodes.

10. The system of claim 1 wherein said probe comprises at least 64 electrodes.

11. The system of claim 1 comprising a plurality of said probes.

12. The system of claim 2 wherein said stimulation signal is time modulated such that evoked electrical signals corresponding to each tissue site may be separately monitored.

13. The system of claim 1 wherein said probe comprises a well having a planar base portion, said base portion comprising said plurality of electrodes.

14. A method for monitoring electrophysiological information from a tissue sample comprising:
(a.) placing a sample of tissue on a probe, said probe comprising a plurality of electrodes;
(b.) selecting a first set of electrodes to monitor electrical signals of the tissue;
(c.) automatically selecting a second set of electrodes to monitor electrical signals of the tissue; and
(c.) monitoring the electrical signals.

15. The method of claim 14 further comprising selecting a first set of electrodes to electrically activate such that a stimulating signal is provided to the tissue.

16. The method of claim 15 further comprising selecting a second set of electrodes to electrically activate.

17. The method of claim 16 wherein said first set and second set of electrodes to be activated are activated with identical stimulating signals.

18. The method of claim 17 wherein said first set and second set of electrodes to be activated are activated with different stimulating signals.

19. The method of claim 17 wherein said first set and second set of electrodes to be activated are activated in sequence.

20. The method of claim 19 comprising sequentially applying at least 64 stimulation signals to different sets of electrodes of said probe.

21. The method of claim 14 further comprising amplifying electrical signals sensed by the electrodes being monitored.

22. The method of claim 14 wherein said probe has 16 or more electrodes.

23. The method of claim 21 wherein an amplifier module performs the amplifying and said amplifier module also is configured to distribute stimulation signals to the electrodes.

24. The method of claim 23 wherein a controller configures the amplifier module and controls the stimulation signals supplied to the amplifier module.

25. The method of claim 24 wherein a computer provides commands to the controller that the controller implements to automatically monitor and stimulate a plurality of tissue sites of the tissue sample.

26. The method of claim 24 comprising a plurality of probes each adapted to contain a tissue sample.

27. The method of claim 26 wherein said tissue sample is a tissue slice.

28. The method of claim 15 wherein said first set of electrodes to activate is activated by applying a voltage potential between at least two electrodes.

29. An electrophysiological information monitoring system comprising:
at least one probe means for holding a tissue slice and for monitoring electrical activity of one or more tissue sites of the tissue slice; and
a control means for selecting tissue sites to monitor, said control means being connected to said at least one probe means.

30. The system of claim 29 wherein said control means is further configured to automatically select an electrical stimulation signal to send to said at least one probe means.

31. The system of claim 30 further comprising an amplifier means for each probe means, said amplifier means being connected between said control means and probe means, said amplifier means adapted to amplify electrical signals evoked from each tissue site.

32. The system of claim 31 wherein said amplifier means is further configured to automatically distribute said stimulation signal to at least one electrode of the probe means.

33. The system of claim 32 wherein said probe means comprises at least 64 electrodes.

34. The system of claim 32 wherein stimulation signals are sent sequentially to said electrodes for evoking electrical signals from said tissue.

35. The system of claim 32 further comprising a computer connected to said control means to supply commands to said control means for selecting said stimulation signal, said computer also configured to record said electrical signals evoked from each tissue site.

36. The system of claim 32 comprising a plurality of probes.

37. A system for monitoring electrophysiological information comprising:
a plurality of probes, each of said probes being adapted to hold a tissue slice and each of said probe means comprising a plurality of electrodes for monitoring electrical activity of the tissue slice;
a daughter amplifier module for each probe, each said daughter amplifier module being configured to amplify signals sensed at said electrodes;
a plurality of daughter controllers, each said daughter controller configured to control one or more daughter amplifiers;
a primary controller configured to control said daughter controllers and to select electrodes to monitor and to activate; and
a computer for delivering instructions to said primary controller and for recording information sent to said computer such that electrical activity of a plurality of tissue slices may be monitored.

38. The system of claim 37 further comprising a primary amplifier module for each of said daughter controllers, said primary amplifier module being configured by said primary controller and for managing an associated daughter controller.

39. The system of claim 37 wherein said probe comprises at least 16 microelectrodes.

40. The system of claim 37 comprising between 4-10 daughter controllers.

41. The system of claim 39 comprising between 4-10 probes for each daughter controller.

42. The system of claim 38 comprising an integrated housing containing said primary controller, said primary amplifier modules, said daughter controllers, and said daughter amplifier modules.

43. The system of claim 37 wherein the primary controller is configured to electrically stimulate and monitor the tissue slices by time-multiplexing.
